(19) United States
(12) Patent Application Publication
(10) Pub. No.: US 2017/0165379 A1
(43) Pub. Date: Jun. 15, 2017

(54) Endoscopic Mucosal Lift Kit

(71) Applicant: Stephanie Ann Wellington, Charlotte, NC (US)

(72) Inventor: Stephanie Ann Wellington, Charlotte, NC (US)

(21) Appl. No.: 15/083,813

(22) Filed: Mar. 29, 2016

Related U.S. Application Data
(63) Continuation-in-part of application No. 14/964,702, filed on Dec. 10, 2015.

(51) Int. Cl. A61K 49/00; A61K 9/00; A61K 9/08; A61K 47/36

(52) U.S. Cl. CPC A61K 49/006; A61K 47/36; A61K 9/0019; A61K 9/08; A61B 17/32056

(57) ABSTRACT

Method and Product; For use in the human digestive tract for endoscopic submucosal injection, as an aid in identifying the extent and borders of flat mucosal polyps (neoplasia) and creating a dynamic gastrointestinal tract mucosa lift of the surrounding mucosa for a safe and through snare polypectomy.

### BACKGROUND

Presently to remove a large sessile colonic polyp the assistant to the endoscopist must mix together Indigo carmine and a volume expander (such as hetastarch), draw up the contents, transfer the contents to a syringe and flush the injector needle. This can take up to 5 minutes which is valuable time in a sedated patient with potential loss of the operative field or the polypectomy site. Indigo carmine is a dye. Hetastarch is a 0.9% saline solution used in hospitals as a volume expander for things such as dehydration and is presently an industry standard for a mucosal lift technique for a colonic polypectomy, especially in the ascending colon to avoid mucosal perforation.

A colonic mucosal perforation is a major complication from this procedure and generally requires urgent surgical intervention for repair. The injected fluid serves as a safety cushion by increasing the distance between the mucosa and the muscle layer and serosa, thereby at least theoretically decreasing risk of perforation. The addition of dye also helps for the identification of the deep muscle layers (muscularis mucosa) with highlights of the surrounding mucosa. The hetastarch causes the subepithial lift, the methylene blue and indigo carmine are currently used industry standards as contrast agents to visually differentiate the various mucosal layers to the endoscopist.

In addition, indigo carmine and methylene blue currently is used to identity ureteral orifices during cystoscopy and is commonly used in obstetrics and gynecology as a marker dye for: backfilling the bladder as a test for bladder injury, in the uterine cavity to test the patency of the fallopian tubes, in test for premature rupture of membranes in the amniotic fluid compartment and during twin pregnancies to mark the amniotic fluid of one twin.

Currently, medical personnel have to mix a premeasure ampule of indigo carmine dye in a large volume of hetastarch (usually 500 ml) for single use only, when only 10 mls is usually needed for submucosal injection. Once the bag is opened it has to be used within 12 hours by hospital infection control policies. The remainder of the bag is wasted. This is time consuming process during a real-time procedure with the mixture urgently needed. Even having the two unmixed substances on hand is inefficient, since the patient is sedated, the Endoscopist can lose visibility and positioning of the endoscope for the intended polypectomy during this crucial mixing time of several minutes. Having this sterile solution in premixed and readily available to use in small non-luer and luer lock 10 ml syringes would be beneficial for direct patient care and cost efficiency. The waste is created because once opened, continued sterility becomes an issue and its inherent infectious disease risk. A luer lock compatible syringe of this standard premixed sterile solution in smaller volumes (10 mls) that would be readily available would be ideal for snare polypectomy lift technique. This would especially be useful in the ascending or right colon where the colonic wall is thinner, thus adding a safety margin of tissue depth to avoid colonic wall perforation. Using a “hot” snare with a blended electric current in millimeter colonic wall thickness can be hazardous. A sustained volume expansion of the colonic wall, unlike 0.9% normal saline that is currently used with its rapid dissipation, would be a welcomed and safer adjunct to the technique of gastrointestinal endoscopic polypectomy.

### PRODUCT SUMMARY

There is a need for a small volume, readily available, single use, and prefilled sterile syringe with

Indigo carmine combined with hetastarch (6% Hetastarch in 0.9% Sodium Chloride) for submucosal endoscopic injection for polypectomy. There is need for a single use complete kit with the injector needle, and specific concentrations of indigo carmine and Hetastarch. This may also be done with the concentrations in a single syringe but need to be separated in a crack and shake syringe, time is important with a polypectomy. Specifically, for 10 and 20 ml premixed syringes of those liquids combined to stop the waste and save on time for these procedures in hospitals and independent Endoscopy Centers in the US and internationally. This patent would be for a kit including the prefilled 20 ml luer-lok syringe of 6% hetastarch in 0.9% normal saline solution. Included in the kit would be a 5 cc vial of indigo carmine and a 5 cc vial of methylene blue concentration, for a final dye concentration of 0.004% when mixed. This assembled kit would be immediately available for the endoscopist for lifting of a large and difficult colonic mucosal polyp. An inclusion of a one-time use through the scope injector needle would be included in the kit.

Thus, the patented product would include: a prefilled 20 ml luer-lok syringe with the endoscopic injector needle and contrast agents as a “ready to use” product. The injector needle would be from a commercially available supplier with the following dimensions: working length; 2300 cm, with a 4 mm length middle beveled needle of 26 gauge (0.4 mm). The included contrast agents (methylene blue and/or indigo carmine would be in hermetically sealed 5 ml vials/ampules to avoid loss of potency and extend shelf life to be opened and mixed in the 20 ml syringe of the 6% hetastarch. As an alternative to the separate solution kit, another option would be a kit that would contain both of the above solutions in a dual chambered syringe but in separate chambers. When mixed it would achieve the same concentrations for injection as above (final dye concentration of 0.004%). One chamber would contain methylene blue and/or indigo carmine, the other the 6% hetastarch solution. The contents of which would be internally mixed immediately prior to use. This would be accomplished with the rupture of an internal separation within the same syringe, thus maintaining sterility and the appropriate concentration.

This product could also be applied to urologic and obstetric surgery, either open or by laparoscopic use, as mentioned above.

### BRIEF DESCRIPTION

The kit would have three options for marketing;

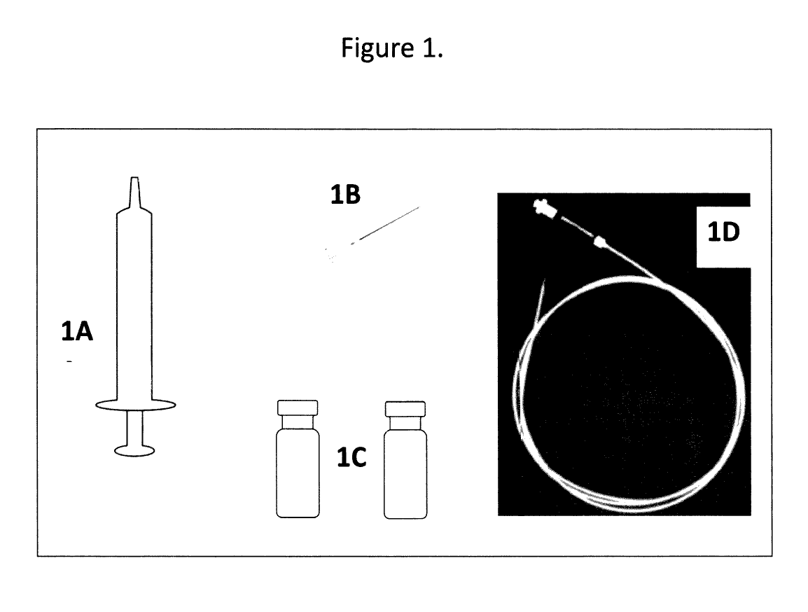

FIG. 1

1A.) 20 ml syringe Luer-Lok™ prefilled syringe with hetastarch in 9% normal saline.

1B.) 22 gauge needle to draw up dye from vials.

1C.) Two vials: Either 5 ml ampule of methylene blue or 5 ml ampule of indigo carmine (At this point due to non-availability of indigo carmine, may need to change name to “Mucosal Lifting Kit” since indigo carmine will unlikely be part of the kit now).

1D.) Endoscopic injector needle

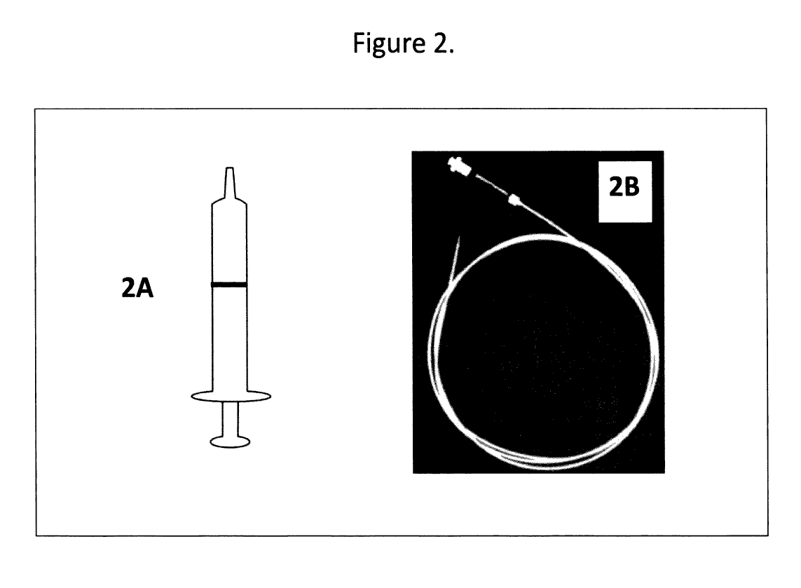

FIG. 2

2A. Dual Chamber Luer-Lok™ prefilled syringe (20 ml) containing 1 ml of indigo carmine (UI methylene blue) and 19 ml of hetastarch in 9% normal saline. To be mixed immediately prior to use.

2R. Endoscopic injector needle

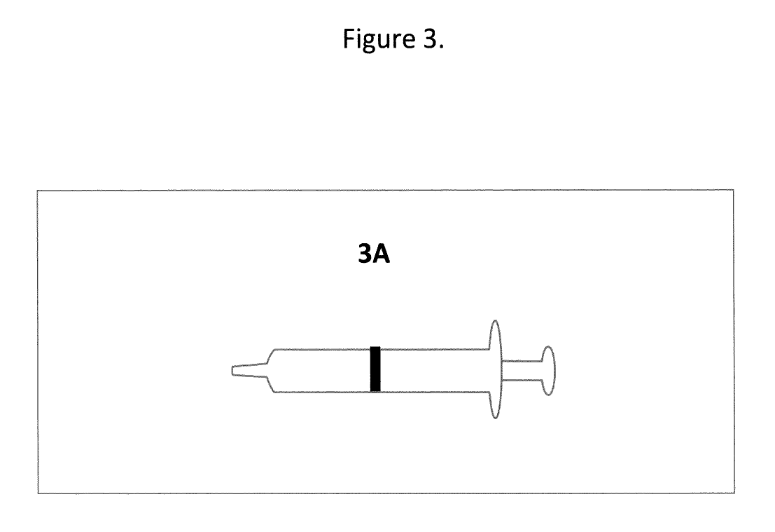

FIG. 3

3A) Dual Chamber prefilled Luer-Lok™ syringe (20 ml) containing 1 ml of indigo carmine (or methylene blue) and 19 ml of hetastarch in 9% normal saline. To be mixed immediately prior to use.

1. As described in trade Endoscopic scientific journals and practiced by Gastroenterologists this endoscopic lift technique is widely accepted. Having “ready to go” injector kits with the appropriate concentration of indigo carmine and or methylene blue, with the appropriate volume of hetastarch would save time and be cost effective for a ‘high risk” colonic polypectomy and possibly other procedures.
